SEQUENCE LISTING (257 sequences)

SEQ ID NO: 1 - DNA, 400 bp, Rattus rattus
tcaatccacacgtgagtcttgctcaaaaggaagagggttcccagcatctccctgaacaaacgtttcattctgtgtttttccgttctagtccaggacacacagatgtaaacctggagatgagacctgaggagaggtggaaccatgttggccttgtacaacgtgaagaagcagattctgtcttggaagagcctatcaacgtagatgaggaagatggaggtcttcaaatctgccgtgtatgtggggacaaggccaatggctatcacttcaatgtcatgacctgtgaaggatgtaagggatttttcaggtatgatcacctacctatcaaccttcatgcacttgctgtattgacacactaggtcacatctaacttgggtttttccgtcaacttgctggacaatgt

SEQ ID NO: 2 - DNA, 28 bp, Rattus rattus
catacacggcagatttgaagacctccat

SEQ ID NO: 3 - DNA, 28 bp, Rattus rattus
gggacaaggccaatggctatcacttcaa

SEQ ID NO: 4 - DNA, 28 bp, Rattus rattus
tggtagccaagacaagaacttctgctta

SEQ ID NO: 5 - DNA, 28 bp, Rattus rattus
aagtagaaagatatgaaataaaaggtat

SEQ ID NO: 6 - DNA, 28 bp, Rattus rattus
ggtagacgggaggcacagaaggagactt

SEQ ID NO: 7 - DNA, 28 bp, Rattus rattus
gttctccaagaagtgagagtggatgatg

SEQ ID NO: 8 - DNA, 28 bp, Rattus rattus
gaaccaggtagacgggaggcacagaagg

SEQ ID NO: 9 - DNA, 28 bp, Rattus rattus
caagaagtgagagtggatgatgttattt

SEQ ID NO: 10 - DNA, 28 bp, Rattus rattus
ttggagaaccaggtagacgggaggcaca

SEQ ID NO: 11 - DNA, 28 bp, Rattus rattus
aagtgagagtggatgatgttatttttct

SEQ ID NO: 12 - DNA, 28 bp, Rattus rattus
gttaaataaggtatggatagaaaaataa

SEQ ID NO: 13 - DNA, 28 bp, Rattus rattus
cacatacagaagagtgaaccccactggc

SEQ ID NO: 14 - DNA, 28 bp, Rattus rattus
ggttgcctgctgctcagctgccagtggg

SEQ ID NO: 15 - DNA, 28 bp, Rattus rattus
catcataggcagtgagaagggggctttt

SEQ ID NO: 16 - DNA, 28 bp, Rattus rattus
tgatggggttgcctgctgctcagctgcc

SEQ ID NO: 17 - DNA, 28 bp, Rattus rattus
aggcagtgagaagggggctttttgcagg

SEQ ID NO: 18 - DNA, 28 bp, Rattus rattus
gaatagcagggcaactgacattagctca

SEQ ID NO: 19 - DNA, 28 bp, Rattus rattus
tgcaccagggagggtggataaccaaggg

SEQ ID NO: 20 - DNA, 28 bp, Rattus rattus
gcaaggaatagcagggcaactgacatta

SEQ ID NO: 21 - DNA, 28 bp, Rattus rattus
ccagggagggtggataaccaagggaatc

SEQ ID NO: 22 - DNA, 28 bp, Rattus rattus
ccctggtgcaaggaatagcagggcaact

SEQ ID NO: 23 - DNA, 28 bp, Rattus rattus
gggtggataaccaagggaatcaactgaa

SEQ ID NO: 24 - DNA, 28 bp, Rattus rattus
tatatagggatgtctgattgataaaagc

SEQ ID NO: 25 - DNA, 28 bp, Rattus rattus
aagaaaaagctccagatgtctgaaataa

SEQ ID NO: 26 - DNA, 28 bp, Rattus rattus
ctgtcctctagtctgattttcctccaaa

SEQ ID NO: 27 - DNA, 28 bp, Rattus rattus
agcaacagattggagaactggcaagaac

SEQ ID NO: 28 - DNA, 28 bp, Rattus rattus
ttgctgcagttgccaaaagagttcttgc

SEQ ID NO: 29 - DNA, 28 bp, Rattus rattus
ccagacgtctgtatgaaacacctaaaac

SEQ ID NO: 30 - DNA, 28 bp, Rattus rattus
gggccaggatgtatgaacagtcatgcag

SEQ ID NO: 31 - DNA, 28 bp, Rattus rattus
atccagttcaccagaccaggtcttcaag

SEQ ID NO: 32 - DNA, 28 bp, Rattus rattus
gaactggatgtgggccaggatgtatgaa

SEQ ID NO: 33 - DNA, 28 bp, Rattus rattus
cagaccaggtcttcaagagactcctctg

SEQ ID NO: 34 - DNA, 28 bp, Rattus rattus
gtgtggctctgcccatatgatctcaatg

SEQ ID NO: 35 - DNA, 28 bp, Rattus rattus
ctggtgcatcatcaggacaaaaagctga

SEQ ID NO: 36 - DNA, 28 bp, Rattus rattus
agtctccatatgcgtgggataagataaa

SEQ ID NO: 37 - DNA, 28 bp, Rattus rattus
tataagataaggggaatgaaaaggacct

SEQ ID NO: 38 - DNA, 28 bp, Rattus rattus
aagagtctccatatgcgtgggataagat

SEQ ID NO: 39 - DNA, 28 bp, Rattus rattus
aagataaggggaatgaaaaggaccttta

SEQ ID NO: 40 - DNA, 28 bp, Rattus rattus
tataagagtctccatatgcgtgggataa

SEQ ID NO: 41 - DNA, 28 bp, Rattus rattus
gataaggggaatgaaaaggacctttagt

SEQ ID NO: 42 - DNA, 28 bp, Rattus rattus
ataaggggaatgaaaaggacctttagtg

SEQ ID NO: 43 - DNA, 28 bp, Rattus rattus
gtcaggaggataaaagcttaagagcaac

SEQ ID NO: 44 - DNA, 28 bp, Rattus rattus
ctattgttatgtctccaggttcagcaag

SEQ ID NO: 45 - DNA, 28 bp, Rattus rattus
gaagtcaggaggataaaagcttaagagc

SEQ ID NO: 46 - DNA, 28 bp, Rattus rattus
ttgttatgtctccaggttcagcaaggag

SEQ ID NO: 47 - DNA, 28 bp, Rattus rattus
aacaatagaagtcaggaggataaaagct

SEQ ID NO: 48 - DNA, 28 bp, Rattus rattus
gtctccaggttcagcaaggagaacaagt

SEQ ID NO: 49 - DNA, 28 bp, Rattus rattus
catacccgtctccaatggacttgacttg

SEQ ID NO: 50 - DNA, 28 bp, Rattus rattus
catttgacatccagaggtgcaatgatca

SEQ ID NO: 51 - DNA, 28 bp, Rattus rattus
gaagagtcgaacatgcaacaaacattga

SEQ ID NO: 52 - DNA, 28 bp, Rattus rattus
tcagagcacctggaaggccatttctcat

SEQ ID NO: 53 - DNA, 28 bp, Rattus rattus
gggcaacaagtgattttaggaactgttt

SEQ ID NO: 54 - DNA, 28 bp, Rattus rattus
taaaaggagttgagtatatttttcagta

SEQ ID NO: 55 - DNA, 28 bp, Rattus rattus
ctcctaagaaatgagctggaattttact

SEQ ID NO: 56 - DNA, 28 bp, Rattus rattus
ctgctctagaaaaataggtatttgtcat

SEQ ID NO: 57 - DNA, 28 bp, Rattus rattus
aacaagaataggttattcaggaaaatgc

SEQ ID NO: 58 - DNA, 28 bp, Rattus rattus
ctgtaaaggaatgggcgcatcttatagg

SEQ ID NO: 59 - DNA, 28 bp, Rattus rattus
gatgaatggggaaagcctccaatagaga

SEQ ID NO: 60 - DNA, 28 bp, Rattus rattus
ctgaagagcacaaggctgaattacaatt

SEQ ID NO: 61 - DNA, 28 bp, Rattus rattus
gtgatgaatggggaaagcctccaataga

SEQ ID NO: 62 - DNA, 28 bp, Rattus rattus
aagagcacaaggctgaattacaattgga

SEQ ID NO: 63 - DNA, 28 bp, Rattus rattus
aagaatgggcaactgattctacaaaatt

SEQ ID NO: 64 - DNA, 28 bp, Rattus rattus
catccttggaagccaagaatagatgcta

SEQ ID NO: 65 - DNA, 28 bp, Rattus rattus
gtgaagaatgggcaactgattctacaaa

SEQ ID NO: 66 - DNA, 28 bp, Rattus rattus
ccttggaagccaagaatagatgctagaa

SEQ ID NO: 67 - DNA, 28 bp, Rattus rattus
ctcctgtggaagggaagctaggagaact

SEQ ID NO: 68 - DNA, 28 bp, Rattus rattus
ttagagcttccctgatgtttgactgtgg

SEQ ID NO: 69 - DNA, 28 bp, Rattus rattus
tctaagactcctgtggaagggaagctag

SEQ ID NO: 70 - DNA, 28 bp, Rattus rattus
cttccctgatgtttgactgtggctctct

SEQ ID NO: 71 - DNA, 28 bp, Rattus rattus
gtcgtgaatgtgggaagaagatttgcag

SEQ ID NO: 72 - DNA, 28 bp, Rattus rattus
gtttcatgtcctaaggacattatctagt

SEQ ID NO: 73 - DNA, 28 bp, Rattus rattus
ataatgtccttaggacatgaaacaagtc

SEQ ID NO: 74 - DNA, 28 bp, Rattus rattus
tagtgccatgtgcgtgactgtgagtttg

SEQ ID NO: 75 - DNA, 28 bp, Rattus rattus
cgcacatggcactagataatgtccttag

SEQ ID NO: 76 - DNA, 28 bp, Rattus rattus
actgtgagtttggaagcctgcgggtcac

SEQ ID NO: 77 - DNA, 28 bp, Rattus rattus
aaagagacagggcctgacactcattgct

SEQ ID NO: 78 - DNA, 28 bp, Rattus rattus
cactggcaccaaagggtggcaggtgttg

SEQ ID NO: 79 - DNA, 28 bp, Rattus rattus
agtgagaaagagacagggcctgacactc

SEQ ID NO: 80 - DNA, 28 bp, Rattus rattus
caccaaagggtggcaggtgttgaatttt

SEQ ID NO: 81 - DNA, 28 bp, Rattus rattus
ccagtgagaaagagacagggcctgacac

SEQ ID NO: 82 - DNA, 28 bp, Rattus rattus
accaaagggtggcaggtgttgaattttg

SEQ ID NO: 83 - DNA, 28 bp, Rattus rattus
tgccagtgagaaagagacagggcctgac

SEQ ID NO: 84 - DNA, 28 bp, Rattus rattus
aaagggtggcaggtgttgaattttggtg

SEQ ID NO: 85 - DNA, 28 bp, Rattus rattus
ggtgccagtgagaaagagacagggcctg

SEQ ID NO: 86 - DNA, 28 bp, Rattus rattus
agggtggcaggtgttgaattttggtgga

SEQ ID NO: 87 - DNA, 28 bp, Rattus rattus
ttggtgccagtgagaaagagacagggcc

SEQ ID NO: 88 - DNA, 28 bp, Rattus rattus
gggtggcaggtgttgaattttggtggat

SEQ ID NO: 89 - DNA, 28 bp, Rattus rattus
ccagcacgcatgatgagtgcttctttgt

SEQ ID NO: 90 - DNA, 28 bp, Rattus rattus
cagttgcctcaggggttgagtgggcttt

SEQ ID NO: 91 - DNA, 28 bp, Rattus rattus
tgtccagcacgcatgatgagtgcttctt

SEQ ID NO: 92 - DNA, 28 bp, Rattus rattus
tgcctcaggggttgagtgggcttttgca

SEQ ID NO: 93 - DNA, 28 bp, Rattus rattus
ccagaagtgcaaaagcccactcaacccc

SEQ ID NO: 94 - DNA, 28 bp, Rattus rattus
aactggtgcttgattcctagacattaca

SEQ ID NO: 95 - DNA, 28 bp, Rattus rattus
tctaggaatcaagcaccagttctccaga

SEQ ID NO: 96 - DNA, 28 bp, Rattus rattus
ttacagtggagatataggtccttgagca

SEQ ID NO: 97 - DNA, 28 bp, Rattus rattus
cagtgccatataagtaagaccaaagttt

SEQ ID NO: 98 - DNA, 28 bp, Rattus rattus
gttcactgacgccacccagaatgttaac

SEQ ID NO: 99 - DNA, 28 bp, Rattus rattus
aacagaagatggcagtggccagtagcag

SEQ ID NO: 100 - DNA, 28 bp, Rattus rattus
gtgttctgggtgttgagaggcacaagga

SEQ ID NO: 101 - DNA, 28 bp, Rattus rattus
actaaacagaagatggcagtggccagta

SEQ ID NO: 102 - DNA, 28 bp, Rattus rattus
tctgggtgttgagaggcacaaggaccca

SEQ ID NO: 103 - DNA, 28 bp, Rattus rattus
cttcagacctttgggaacctgggtcctt

SEQ ID NO: 104 - DNA, 28 bp, Rattus rattus
tcctcccggaccctggggcttgcccttc

SEQ ID NO: 105 - DNA, 28 bp, Rattus rattus
gaggatactgatgtggggtctgaggcta

SEQ ID NO: 106 - DNA, 28 bp, Rattus rattus
gctacttggaggagcacgtgagcaaaga

SEQ ID NO: 107 - DNA, 28 bp, Rattus rattus
aagaggatactgatgtggggtctgaggc

SEQ ID NO: 108 - DNA, 28 bp, Rattus rattus
tacttggaggagcacgtgagcaaagagg

SEQ ID NO: 109 - DNA, 28 bp, Rattus rattus
agcaagaggatactgatgtggggtctga

SEQ ID NO: 110 - DNA, 28 bp, Rattus rattus
cttggaggagcacgtgagcaaagaggct

SEQ ID NO: 111 - DNA, 28 bp, Rattus rattus
tagcaagaggatactgatgtggggtctg

SEQ ID NO: 112 - DNA, 28 bp, Rattus rattus
tggaggagcacgtgagcaaagaggctaa

SEQ ID NO: 113 - DNA, 28 bp, Rattus rattus
acttgctgattagatggttagcctcttt

SEQ ID NO: 114 - DNA, 28 bp, Rattus rattus
cagaagctgatggcagaggttggccact

SEQ ID NO: 115 - DNA, 28 bp, Rattus rattus
ttgactggttcgaagtggccaacctctg

SEQ ID NO: 116 - DNA, 28 bp, Rattus rattus
caggtggtggaatcggtggctaatgtca

SEQ ID NO: 117 - DNA, 28 bp, Rattus rattus
gttgactggttcgaagtggccaacctct

SEQ ID NO: 118 - DNA, 28 bp, Rattus rattus
aggtggtggaatcggtggctaatgtcat

SEQ ID NO: 119 - DNA, 28 bp, Rattus rattus
accacctggttgactggttcgaagtggc

SEQ ID NO: 120 - DNA, 28 bp, Rattus rattus
aatcggtggctaatgtcatcggagccat

SEQ ID NO: 121 - DNA, 28 bp, Rattus rattus
ttcacgaggttgagcatctcctcgctct

SEQ ID NO: 122 - DNA, 28 bp, Rattus rattus
gcagcaaggactttgtggagaatgtcac

SEQ ID NO: 123 - DNA, 28 bp, Rattus rattus
gggttgggcaggtagcgcaggaccggaa

SEQ ID NO: 124 - DNA, 28 bp, Rattus rattus
ccctcaagaggtttaagaacttcaatga

SEQ ID NO: 125 - DNA, 28 bp, Rattus rattus
gctgggttgggcaggtagcgcaggaccg

SEQ ID NO: 126 - DNA, 28 bp, Rattus rattus
ctcaagaggtttaagaacttcaatgata

SEQ ID NO: 127 - DNA, 28 bp, Rattus rattus
agggctgggttgggcaggtagcgcagga

SEQ ID NO: 128 - DNA, 28 bp, Rattus rattus
aagaggtttaagaacttcaatgataact

SEQ ID NO: 129 - DNA, 28 bp, Rattus rattus
gtcttgatagtgttcctggactgttttc

SEQ ID NO: 130 - DNA, 28 bp, Rattus rattus
caacaaggtgagactgagaggcagactg

SEQ ID NO: 131 - DNA, 28 bp, Rattus rattus
tctgtgtgagggtctcagaagctgtgga

SEQ ID NO: 132 - DNA, 28 bp, Rattus rattus
tacatggaggcaaaacaaggttgaacta

SEQ ID NO: 133 - DNA, 28 bp, Rattus rattus
agaatgcgggataacagaatggtcataa

SEQ ID NO: 134 - DNA, 28 bp, Rattus rattus
gctaaaagggctttgtatgcaaaaggta

SEQ ID NO: 135 - DNA, 28 bp, Rattus rattus
agagaatgcgggataacagaatggtcat

SEQ ID NO: 136 - DNA, 28 bp, Rattus rattus
ctaaaagggctttgtatgcaaaaggtac

SEQ ID NO: 137 - DNA, 28 bp, Rattus rattus
gcagagaatgcgggataacagaatggtc

SEQ ID NO: 138 - DNA, 28 bp, Rattus rattus
aaagggctttgtatgcaaaaggtacctt

SEQ ID NO: 139 - DNA, 28 bp, Rattus rattus
aagtcatgtaattagctcatataataaa

SEQ ID NO: 140 - DNA, 28 bp, Rattus rattus
ttggtgagtaatcatggctaatattact

SEQ ID NO: 141 - DNA, 28 bp, Rattus rattus
ttggtgagtaatcatggctaatattact

SEQ ID NO: 142 - DNA, 28 bp, Rattus rattus
caagagcctggcttgccataaaaatgac

SEQ ID NO: 143 - DNA, 28 bp, Rattus rattus
tggatgttgctatgtaacagttgggtag

SEQ ID NO: 144 - DNA, 28 bp, Rattus rattus
cagacaagagcctggcttgccataaaaa

SEQ ID NO: 145 - DNA, 28 bp, Rattus rattus
atgttgctatgtaacagttgggtagagc

SEQ ID NO: 146 - DNA, 28 bp, Rattus rattus
tagcaacatccagacaagagcctggctt

SEQ ID NO: 147 - DNA, 28 bp, Rattus rattus
taacagttgggtagagccaagaaggaat

SEQ ID NO: 148 - DNA, 28 bp, Rattus rattus
taacagttgggtagagccaagaaggaat

SEQ ID NO: 149 - DNA, 28 bp, Rattus rattus
acccaaagttagcttcttagcattcctt

SEQ ID NO: 150 - DNA, 28 bp, Rattus rattus
cacccctggttagttacacagatttgtg

SEQ ID NO: 151 - DNA, 28 bp, Rattus rattus
gggtgggacccaaagttagcttcttagc

SEQ ID NO: 152 - DNA, 28 bp, Rattus rattus
ggttagttacacagatttgtggtagtag

SEQ ID NO: 153 - DNA, 28 bp, Rattus rattus
taaccaggggtgggacccaaagttagct

SEQ ID NO: 154 - DNA, 28 bp, Rattus rattus
taaccaggggtgggacccaaagttagct

SEQ ID NO: 155 - DNA, 28 bp, Rattus rattus
ttacacagatttgtggtagtagaaagag

SEQ ID NO: 156 - DNA, 28 bp, Rattus rattus
caaatctgtgtaactaaccaggggtggg

SEQ ID NO: 157 - DNA, 28 bp, Rattus rattus
ggtagtagaaagaggaagaacagttttc

SEQ ID NO: 158 - DNA, 28 bp, Rattus rattus
ccacaaatctgtgtaactaaccaggggt

SEQ ID NO: 159 - DNA, 28 bp, Rattus rattus
gtagaaagaggaagaacagttttcactt

SEQ ID NO: 160 - DNA, 28 bp, Rattus rattus
ggggaacataggccagaaggaaaccggg

SEQ ID NO: 161 - DNA, 28 bp, Rattus rattus
aatgtgatggtattgcatgtgggacagg

SEQ ID NO: 162 - DNA, 28 bp, Rattus rattus
gaggggaacataggccagaaggaaaccg

SEQ ID NO: 163 - DNA, 28 bp, Rattus rattus
atgtgatggtattgcatgtgggacaggc

SEQ ID NO: 164 - DNA, 28 bp, Rattus rattus
cattgaggggaacataggccagaaggaa

SEQ ID NO: 165 - DNA, 28 bp, Rattus rattus
atggtattgcatgtgggacaggcataag

SEQ ID NO: 166 - DNA, 28 bp, Rattus rattus
ctctagaaatgagtgcagagagttctgg

SEQ ID NO: 167 - DNA, 28 bp, Rattus rattus
gggaaacagccattggactcccctccag

SEQ ID NO: 168 - DNA, 28 bp, Rattus rattus
gtccaatggctgtttcccctctctagaa

SEQ ID NO: 169 - DNA, 28 bp, Rattus rattus
ccctccaggttcctgactccccctgctg

SEQ ID NO: 170 - DNA, 28 bp, Rattus rattus
caggaacctggaggggagtccaatggct

SEQ ID NO: 171 - DNA, 28 bp, Rattus rattus
tccccctgctggcagtataccagcggtg

SEQ ID NO: 172 - DNA, 28 bp, Rattus rattus
gagtcaggaacctggaggggagtccaat

SEQ ID NO: 173 - DNA, 28 bp, Rattus rattus
ccctgctggcagtataccagcggtgcct

SEQ ID NO: 174 - DNA, 28 bp, Rattus rattus
cagagaggaagcagagcaaagtctgaaa

SEQ ID NO: 175 - DNA, 28 bp, Rattus rattus
ggcatgccagcggcccttggttacaccc

SEQ ID NO: 176 - DNA, 28 bp, Rattus rattus
gggggggtgtaaccaagggccgctggca

SEQ ID NO: 177 - DNA, 28 bp, Rattus rattus
cacccctggtagagaggcaggatgtagc

SEQ ID NO: 178 - DNA, 28 bp, Rattus rattus
gggtggggggggggtgtaaccaagggcc

SEQ ID NO: 179 - DNA, 28 bp, Rattus rattus
tggtagagaggcaggatgtagccctata

SEQ ID NO: 180 - DNA, 28 bp, Rattus rattus
aggggtggggggggggtgtaaccaaggg

SEQ ID NO: 181 - DNA, 28 bp, Rattus rattus
gtagagaggcaggatgtagccctatatg

SEQ ID NO: 182 - DNA, 28 bp, Rattus rattus
accaggggtggggggggggtgtaaccaa

SEQ ID NO: 183 - DNA, 28 bp, Rattus rattus
agaggcaggatgtagccctatatgcaat

SEQ ID NO: 184 - DNA, 28 bp, Rattus rattus
tctaccaggggtggggggggggtgtaac

SEQ ID NO: 185 - DNA, 28 bp, Rattus rattus
ggcaggatgtagccctatatgcaatctc

SEQ ID NO: 186 - DNA, 28 bp, Rattus rattus
cccaagatgtgcagaaactgaccagaga

SEQ ID NO: 187 - DNA, 28 bp, Rattus rattus
tttgtgcattatgcaagaggatgggaaa

SEQ ID NO: 188 - DNA, 28 bp, Rattus rattus
ccttagagacaatagactaactctgcac

SEQ ID NO: 189 - DNA, 28 bp, Rattus rattus
aataaaaagctcataggaacaacacaca

SEQ ID NO: 190 - DNA, 28 bp, Rattus rattus
gtggcagagctctggaacatggcttcat

SEQ ID NO: 191 - DNA, 28 bp, Rattus rattus
tggaggttttttggagtcagcctgggat

SEQ ID NO: 192 - DNA, 28 bp, Rattus rattus
acagggatgattcggttcagagtaaaac

SEQ ID NO: 193 - DNA, 28 bp, Rattus rattus
gctctccaggctgtaaggggcctgagca

SEQ ID NO: 194 - DNA, 28 bp, Rattus rattus
ctcacagggatgattcggttcagagtaa

SEQ ID NO: 195 - DNA, 28 bp, Rattus rattus
tctccaggctgtaaggggcctgagcagc

SEQ ID NO: 196 - DNA, 28 bp, Rattus rattus
ggagagctcacagggatgattcggttca

SEQ ID NO: 197 - DNA, 28 bp, Rattus rattus
gctgtaaggggcctgagcagccttcccg

SEQ ID NO: 198 - DNA, 28 bp, Rattus rattus
cacatggtcaaggaagattaagtagggg

SEQ ID NO: 199 - DNA, 28 bp, Rattus rattus
ccaatggaagggctgctctactgacctc

SEQ ID NO: 200 - DNA, 28 bp, Rattus rattus
gggcacatggtcaaggaagattaagtag

SEQ ID NO: 201 - DNA, 28 bp, Rattus rattus
atggaagggctgctctactgacctccga

SEQ ID NO: 202 - DNA, 28 bp, Rattus rattus
ccattggggcacatggtcaaggaagatt

SEQ ID NO: 203 - DNA, 28 bp, Rattus rattus
gggctgctctactgacctccgaaatggc

SEQ ID NO: 204 - DNA, 28 bp, Rattus rattus
tccagatgttggcatctatgaataaaca

SEQ ID NO: 205 - DNA, 28 bp, Rattus rattus
ggagggccagaaaggactgctgtgaagg

SEQ ID NO: 206 - DNA, 28 bp, Rattus rattus
ttccagatgttggcatctatgaataaac

SEQ ID NO: 207 - DNA, 28 bp, Rattus rattus
agggccagaaaggactgctgtgaagggt

SEQ ID NO: 208 - DNA, 28 bp, Rattus rattus
gtgggtctgaaatgggggcgtctgggag

SEQ ID NO: 209 - DNA, 28 bp, Rattus rattus
acacaaccaaggctaactcctcagccag

SEQ ID NO: 210 - DNA, 28 bp, Rattus rattus
gtagtgggtctgaaatgggggcgtctgg

SEQ ID NO: 211 - DNA, 28 bp, Rattus rattus
caaccaaggctaactcctcagccagcat

SEQ ID NO: 212 - DNA, 28 bp, Rattus rattus
aagttgtgatgatgctggctgaggagtt

SEQ ID NO: 213 - DNA, 28 bp, Rattus rattus
tatatgacgtcgcagagatgtagagaag

SEQ ID NO: 214 - DNA, 28 bp, Rattus rattus
aagaagttgtgatgatgctggctgagga

SEQ ID NO: 215 - DNA, 28 bp, Rattus rattus
atgacgtcgcagagatgtagagaagtcg

SEQ ID NO: 216 - DNA, 28 bp, Rattus rattus
tataagaagttgtgatgatgctggctga

SEQ ID NO: 217 - DNA, 28 bp, Rattus rattus
acgtcgcagagatgtagagaagtcgggg

SEQ ID NO: 218 - DNA, 28 bp, Rattus rattus
tagggaagcatgtggtacccaacaggta

SEQ ID NO: 219 - DNA, 28 bp, Rattus rattus
acggttttgtgggggtccagaagcaggt

SEQ ID NO: 220 - DNA, 28 bp, Rattus rattus
aaaccgtttagggaagcatgtggtaccc

SEQ ID NO: 221 - DNA, 28 bp, Rattus rattus
gtgggggtccagaagcaggttgcctcct

SEQ ID NO: 222 - DNA, 28 bp, Rattus rattus
atggtgacaaagaagcttaggaggcaac

SEQ ID NO: 223 - DNA, 28 bp, Rattus rattus
attccatgacccagcagggatactggtg

SEQ ID NO: 224 - DNA, 28 bp, Rattus rattus
ctttaggggggatggcatctgcatattt

SEQ ID NO: 225 - DNA, 28 bp, Rattus rattus
cacatgatcctgacaggccacaggggtt

SEQ ID NO: 226 - DNA, 28 bp, Rattus rattus
gtgactttaggggggatggcatctgcat

SEQ ID NO: 227 - DNA, 28 bp, Rattus rattus
gatcctgacaggccacaggggttcccat

SEQ ID NO: 228 - DNA, 28 bp, Rattus rattus
ctgtggcctgtcaggatcatgtgacttt

SEQ ID NO: 229 - DNA, 28 bp, Rattus rattus
gttcccatggatgggaggacatggactg

SEQ ID NO: 230 - DNA, 28 bp, Rattus rattus
acaggtggaaggtcgttcaagtctcaag

SEQ ID NO: 231 - DNA, 28 bp, Rattus rattus
ttgaagcttcacaggctggagttacctt

SEQ ID NO: 232 - DNA, 28 bp, Rattus rattus
aagtacaggtggaaggtcgttcaagtct

SEQ ID NO: 233 - DNA, 28 bp, Rattus rattus
aagcttcacaggctggagttaccttcct

SEQ ID NO: 234 - DNA, 28 bp, Rattus rattus
ccactgaagtggcttgtgtgggtagctg

SEQ ID NO: 235 - DNA, 28 bp, Rattus rattus
ccttgctgcatactgatagtggccatgg

SEQ ID NO: 236 - DNA, 28 bp, Rattus rattus
caagggccactgaagtggcttgtgtggg

SEQ ID NO: 237 - DNA, 28 bp, Rattus rattus
gcatactgatagtggccatggtgtcagt

SEQ ID NO: 238 - DNA, 28 bp, Rattus rattus
atgcagcaagggccactgaagtggcttg

SEQ ID NO: 239 - DNA, 28 bp, Rattus rattus
tgatagtggccatggtgtcagtcagacc

SEQ ID NO: 240 - DNA, 28 bp, Rattus rattus
actatcagtatgcagcaagggccactga

SEQ ID NO: 241 - DNA, 28 bp, Rattus rattus
gccatggtgtcagtcagaccccatgtct

SEQ ID NO: 242 - DNA, 28 bp, Rattus rattus
gccttatgggcagagatccacggggcct

SEQ ID NO: 243 - DNA, 28 bp, Rattus rattus
atcagcagcttgcacactgaggaagggt

SEQ ID NO: 244 - DNA, 28 bp, Rattus rattus
tgcaagctgctgatttgccttatgggca

SEQ ID NO: 245 - DNA, 28 bp, Rattus rattus
actgaggaagggttcatgttatgtgctt

SEQ ID NO: 246 - DNA, 28 bp, Rattus rattus
tcagtgtgcaagctgctgatttgcctta

SEQ ID NO: 247 - DNA, 28 bp, Rattus rattus
gaagggttcatgttatgtgcttgtcttg

SEQ ID NO: 248 - DNA, 28 bp, Rattus rattus
gtggcattgaaatctccacagaaagcag

SEQ ID NO: 249 - DNA, 28 bp, Rattus rattus
cccatggacaagcaccaccggagagaag

SEQ ID NO: 250 - DNA, 28 bp, Rattus rattus
tgtccatgggagtggcattgaaatctcc

SEQ ID NO: 251 - DNA, 28 bp, Rattus rattus
caccaccggagagaagaaagggcaagaa

SEQ ID NO: 252 - DNA, 28 bp, Rattus rattus
gtggtgcttgtccatgggagtggcattg

SEQ ID NO: 253 - DNA, 28 bp, Rattus rattus
ggagagaagaaagggcaagaacctctga

SEQ ID NO: 254 - DNA, 28 bp, Rattus rattus
ccggtggtgcttgtccatgggagtggca

SEQ ID NO: 255 - DNA, 28 bp, Rattus rattus
gagaagaaagggcaagaacctctgatgt

SEQ ID NO: 256 - protein, 35 aa, Artificial Sequence
  SYNTHESIZED
DNAALTETSSNLSRQSGDLTRRSDTLSQDNANRTK

SEQ ID NO: 257 - protein, 35 aa, Artificial Sequence
  SYNTHESIZED
QSSDLSRRSDALTQDRSDLSRRSDNLSVDRSNLTR

(19) United States
(12) Patent Application Publication
(10) Pub. No.: US 2012/0023599 A1
(43) Pub. Date: Jan. 26, 2012

(54) GENOME EDITING OF CYTOCHROME P450 IN ANIMALS

(75) Inventors: Edward Weinstein, St. Louis, MO (US); Xiaoxia Cui, St. Louis, MO (US); Victoria Brown-Kennerly, St. Louis, MO (US)

(73) Assignee: SIGMA-ALDRICH CO., St. Louis, MO (US)

(21) Appl. No.: 12/842,943

(22) Filed: Jul. 23, 2010

(51) Int. Cl. G01N 33/48; C07H 21/04; C12N 9/16; A01K 67/027; C12N 5/07

(52) U.S. Cl. 800/3; 800/13; 800/14; 800/16; 800/15; 800/17; 800/8; 435/325; 435/196; 536/23.1

(57) ABSTRACT

The present invention provides genetically modified animals and cells comprising edited chromosomal sequences encoding cytochrome P450 (CYP) proteins. In particular, the animals or cells are generated using a zinc finger nuclease-mediated editing process. The invention also provides zinc finger nucleases that target chromosomal sequence encoding CYP proteins, as well as methods of using the genetically modified animals or cells disclosed herein to screen agents for toxicity and other effects.

### FIELD OF THE INVENTION

The invention generally relates to genetically modified animals or cells comprising at least one edited chromosomal sequence encoding a cytochrome P450 (CYP) protein. In particular, the invention relates to the use of a zinc finger nuclease-mediated process to edit chromosomal sequences encoding CYP proteins.

### BACKGROUND OF THE INVENTION

The vast majority of drugs (approximately 91%) fail to successfully proceed through the three phases of drug testing in humans. A majority of those drugs that fail do so because of unforeseen toxicology in human patients, despite the fact that all of these drugs had been tested in animal models and were found to be safe. This is because toxicology testing is performed in animals, and animal proteins differ from the orthologous proteins in humans.

The major proteins involved in drug metabolism are from the family commonly called Cytochrome P450s or CYPs. Cytochrome P450-dependent monooxygenases (CYPs) are a group of enzymes that account for the Phase I metabolism of the majority of clinically used drugs. These enzymes have diverged significantly between species, both in their multiplicity and substrate specificity. As a result, animals and humans have different drug metabolism profiles and exhibit differences in pharmacokinetics, efficacy, and toxicity. Consequently, the outcomes of preclinical studies in animals may not be predictive of the situation in humans. What is needed is a means to test a drug in an animal, with actual human proteins processing the drug. This means the animal would have to have its endogenous CYP proteins removed and replaced with human CYP proteins (such an animal is commonly called “humanized”).

### SUMMARY OF THE INVENTION

One aspect of the present disclosure encompasses a genetically modified animal comprising at least one edited chromosomal sequence encoding a CYP protein.

Another aspect provides a non-human embryo comprising at least one RNA molecule encoding a zinc finger nuclease that recognizes a site in a chromosomal sequence encoding a CYP protein, and, optionally, at least one donor polynucleotide comprising a sequence encoding an ortholog of the CYP protein.

A further aspect encompasses providing a genetically modified cell comprising at least one edited chromosomal sequence encoding a CYP protein.

Yet another aspect provides a zinc finger nuclease comprising (a) a zinc finger DNA binding domain that binds a chromosomal sequence having at least about 80% sequence identity with SEQ ID NO:2 or SEQ ID NO:3, and (b) a cleavage domain.

Another aspect provides nucleic acid sequence that is bound by a zinc finger nuclease. The nucleic acid sequence has at least about 80% sequence identity with SEQ ID NO:2 or SEQ ID NO:3.

Yet another aspect encompasses a method for assessing the effect of an agent in an animal. The method comprises contacting a genetically modified animal comprising at least one edited chromosomal sequence encoding a CYP protein with the agent, and comparing results of a selected pharmacodynamic or pharmacokinetic parameter to results obtained from contacting a wild-type animal with the same agent. The selected pharmacodynamic or pharmacokinetic parameter is chosen from (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); and (g) efficacy of the agent or its metabolite(s).

Other aspects and features of the disclosure are described more thoroughly below.

### REFERENCE TO COLOR FIGURES

The application file contains at least one figure executed in color. Copies of this patent application publication with color figure will be provided by the Office upon request and payment of the necessary fee.

### BRIEF DESCRIPTION OF THE FIGURES

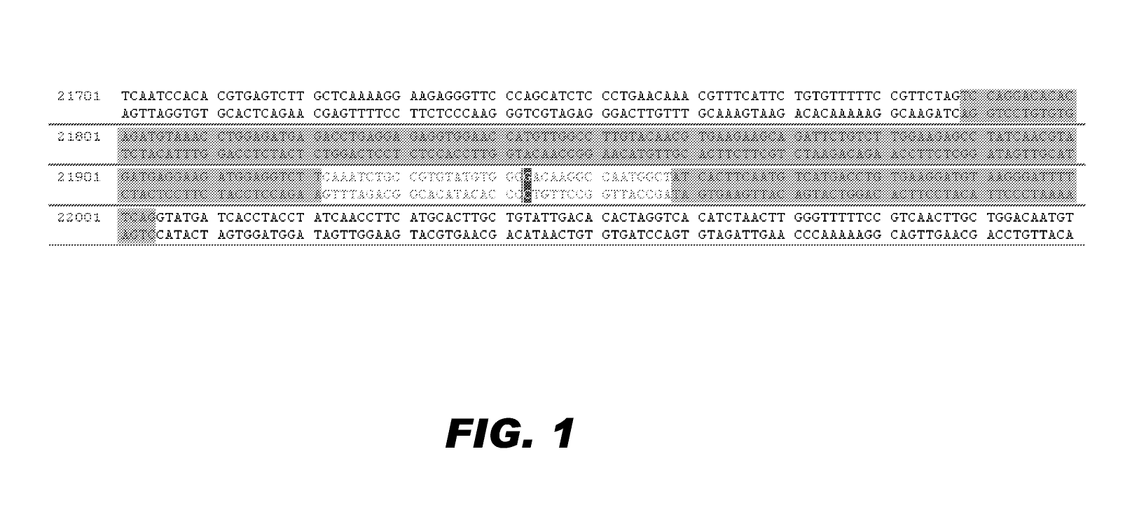

FIG. 1 presents the DNA sequence of an edited pxr locus. Presented is a region of the pxr locus (SEQ ID NO:1) in which there is a one base pair deletion in the target sequence of exon 2. The exon is shown in green; the target site is presented in yellow, and the deletion is shown in dark blue.

### DETAILED DESCRIPTION OF THE INVENTION

The present disclosure provides a genetically modified animal or animal cell comprising at least one edited chromosomal sequence encoding a CYP protein. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the CYP generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding CYP protein using targeted zinc finger nuclease technology is rapid, precise, and highly efficient.

### (I) Genetically Modified Animals

One aspect of the present disclosure provides a genetically modified animal in which at least one chromosomal sequence encoding a CYP protein has been edited. For example, the edited chromosomal sequence may be inactivated such that the sequence is not transcribed and/or a functional CYP protein is not produced. Alternatively, the edited chromosomal sequence may be modified such that it codes for an altered CYP protein. For example, the chromosomal sequence may be modified such that at least one nucleotide is changed and the expressed CYP protein comprises at least changed amino acid residue. The modified CYP protein may have altered substrate specificity, altered enzyme activity, altered kinetic rates, and so forth. Furthermore, the edited chromosomal sequence encoding a CYP protein may comprise an integrated sequence and/or a sequence encoding an orthologous CYP protein may be integrated into the genome of the animal. The genetically modified animal disclosed herein may be heterozygous for the edited chromosomal sequence encoding a CYP protein. Alternatively, the genetically modified animal may be homozygous for the edited chromosomal sequence encoding a CYP protein.

In one embodiment, the genetically modified animal may comprise at least one inactivated chromosomal sequence encoding a CYP protein. The inactivated chromosomal sequence may include a deletion mutation (i.e., deletion of one or more nucleotides), an insertion mutation (i.e., insertion of one or more nucleotides), or a point mutation (i.e., substitution of a single nucleotide for another nucleotide). The deletion, insertion, or point mutation may lead to a frame shift mutation or a splice site mutation such that at least one premature stop codon is introduced. As a consequence of the mutation, the targeted chromosomal sequence is inactivated and a functional CYP protein is not produced. Such an animal may be termed a “knockout.” In an iteration of knockout animals disclosed herein, no exogenously sequence is introduced.

In another embodiment, the genetically modified animal may comprise at least one chromosomally integrated sequence encoding an orthologous CYP protein. Typically, the expressed orthologous protein will be a wild-type CYP protein, but altered versions of the protein also may be introduced. The sequence encoding an orthologous CYP protein may be integrated into the chromosome of the animal in a targeted manner or a random manner. Furthermore, the sequence encoding an orthologous CYP protein may be integrated into any chromosomal sequence such that the chromosomal sequence is inactivated. For example, the sequence may be integrated into a chromosomal sequence encoding a CYP protein such that the chromosomal sequence is inactivated and the CYP is not produced. The chromosomally integrated sequence encoding the ortholog, however, may be expressed. Accordingly, the chromosomally integrated sequence encoding the orthologous CYP protein may be under the control of an endogenous promoter or it may be operably linked to its native promoter or a heterologous promoter. The type of promoter generally will determine the level of expression of the orthologous CYP protein. Alternatively, a sequence encoding an orthologous CYP protein may be integrated into a chromosomal sequence without affecting gene expression. For example, a sequence encoding an orthologous CYP protein may be integrated into a “safe harbor” locus, such as the Rosa26 locus, HPRT locus, or AAVS1 locus. The sequence integrated into a safe harbor locus generally will be operably linked to its native promoter or a heterologous promoter. Animals comprising a chromosomally integrated sequence typically are called “knock-in” animals, and it should be understood that, in such an iteration of the animal, no selectable marker is present.

In an additional embodiment, the genetically modified animal may be a “humanized” animal comprising at least one chromosomally integrated sequence encoding a functional human CYP related protein. Generally, the orthologous sequence in the “humanized” animal is inactivated such that no functional protein is made and the “humanized” animal comprises at least one chromosomally integrated sequence encoding the human CYP protein. For example, a humanized animal may comprise an inactivated cyp1A2 sequence and a chromosomally integrated human CYP1A2 sequence. Those of skill in the art appreciate that “humanized” animals may be generated by crossing a knock out animal with a knock in animal comprising the chromosomally integrated sequence, as detailed below in section (III)(f).

In yet another embodiment, the genetically modified animal may comprise at least one edited chromosomal sequence encoding a CYP protein such that the expression pattern of the protein is altered. For example, regulatory regions controlling the expression of the protein, such as a promoter or transcription binding site, may be altered such that the CYP protein is over-produced, or the tissue-specific or temporal expression of the protein is altered, or a combination thereof. Alternatively, the expression pattern of the CYP protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence of interest. The genetically modified animal comprising the lox-flanked chromosomal sequence may then be crossed with another genetically modified animal expressing Cre recombinase. Progeny animals comprising the lox-flanked chromosomal sequence and the Cre recombinase are then produced, and the lox-flanked chromosomal sequence of interest is recombined, leading to deletion or inversion of the chromosomal sequence of interest. Expression of Cre recombinase may be temporally and conditionally regulated to effect temporally and conditionally regulated recombination of the chromosomal sequence encoding the protein of interest.

(a) animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In another iteration of the invention, the animal does not comprise a genetically modified mouse. In each of the foregoing iterations of suitable animals for the invention, the animal does not include exogenously introduced, randomly integrated transposon sequences.

### (b) CYP Proteins

CYP proteins are members of a superfamily comprising a large number of enzymes involved in the metabolism of endogenous and exogenous substances. In particular, CYP enzymes catalyze the oxidation of organic substances. The substrates of CYP enzymes include metabolic intermediates such as lipids and steroidal hormones, as well as xenobiotic substances such as active pharmaceutical ingredients, drugs, toxins, and other chemicals.

Members of several CYP families (e.g., CYP1, CYP2, and CYP3) play key roles in the metabolism of xenobiotic substances. In humans, these CYP enzymes include CYP1A1, CYP1A2, CYP1B, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, and CYP3A43. Suitable Cyp proteins in rat include Cyp1A1, Cyp1A2, Cyp1B1, Cyp2A1, Cyp2A2, Cyp2A3, Cyp2B1, Cyp2B2, Cyp2B3, Cyp2B8, Cyp2B12, Cyp2B14, Cyp2B15, Cyp2B16P, Cyp2C6, Cyp2C6P, Cyp2C7, Cyp2C11, Cyp2C12, Cyp2C13, Cyp2C22, Cyp2C23, Cyp2C24, Cyp2D1, Cyp2D2, Cyp2D3, Cyp2D4, Cyp2D5, Cyp2D18, Cyp2E1, Cyp2G1, Cyp2J3, Cyp2J3P1, Cyp3J3P2, Cyp2J4, Cyp3A1, Cyp3A2, Cyp3A9, Cyp3A18, and Cyp3A23. Suitable Cyp proteins in rabbit include Cyp1A1, Cyp1A2, Cyp2A10, Cyp2A11, Cyp2B4, Cyp2B5, Cyp2C1, Cyp2C2, Cyp2C3, Cyp2C4, Cyp2C5, Cyp2C14, Cyp2C15, Cyp2C16, Cyp2C30, Cyp2D23, Cyp2D24, Cyp2E1, Cyp2E2, Cyp2G1, Cyp2J1, and Cyp3A6. Those of skill in the art will be familiar with suitable CYP proteins in other organisms.

As used herein, CYP proteins also include PXR, the pregnane X receptor (which is also called NR1I2) and CAR, constitutive androstane receptor (also call NR1I3). PXR and CAR are nuclear receptor whose primary function is to sense the presence of foreign substances and to modulate the expression of proteins (e.g., CYP proteins) involved in the metabolism and clearance of these substances from the body.

The identity of the CYP protein whose chromosomal sequence is edited will vary depending upon the animal and the intended use. Exemplary CYP proteins include those that are functional homologs of human PXR, CYP1A2, CYP2C9, CYP2C19, CYP2D6, CYP2E1, and CYP3A4. Those of skill in the art appreciate that the CYP protein nomenclature differs among different species. This is illustrated in TABLE A, which lists the names of orthologs of the exemplary human CYP proteins.

TABLE A
Different names of orthologous CYP proteins in different species

| human | rat | dog | cow | chimpanzee | chicken |
|---|---|---|---|---|---|
| CYP1A2 | cyp1A2 | CYP1A2 | CYP1A2 | LOC735881 | |
| CYP2C9 | cyp2C11 | | LOC505468 | LOC740956 | CYP2C45 |
| CYP2C19 | cyp2C7 | CYP2C21 | CYP2C19 | | |
| CYP2D6 | cyp2D4 | CYP2D15 | | CYP2D6 | |
| CYP2E1 | cyp2E1 | CYP2E1 | CYP2E1 | LOC450857 | |
| CYP3A4 | cyp3A1 | LOC479740 | CYP3A4 | CYP3A4 | |

In embodiments in which the animal is a rat, exemplary chromosomal sequences that may be edited include pxr (NM_{—}052980), car (NM_{—}022941), cyp1A2 (NM_{—}012541.2), cyp2C11 (NM_{—}019184), cyp2C7 (NM_{—}017158), cyp2D4 (NM_{—}138515), cyp2E1 (NM_{—}0131543), and cyp3A2 (NM_{—}153312). Furthermore, because the functions of some human CYP proteins seem to be shared among several subfamily members in rat, for example, more than one rat cyp sequence may have to be inactivated to achieve complete knock-out. For example, large rat chromosomal regions may be deleted such that no functional homologs of the human CYP protein of interest are made in rat. For example, to eliminate all functional homologs of human CYP2C9 and CYP2C19, the rat chromosomal region from cyp2C11 to cyp2C22 on chromosome 1 may be deleted. Similarly, to eliminate all functional homologs of human CYP2D6, the rat chromosomal region from cyp2D2 to cyp2D4 on chromosome 7 may be deleted. Likewise, to eliminate all functional homologs of human CYP3A4, the rat chromosomal region from cyp3A23/3A1 to cyp3A2 or from cyp3A23/3A1 to cyp3A18 may be deleted.

The number of edited chromosomal sequences in the genetically modified animal can and will vary. For example, genetically modified animals may comprise one, two, three, four, five, six, or seven inactivated chromosomal sequences encoding a CYP protein and zero, one, two, three, four, five, six, or seven chromosomally integrated sequences encoding orthologous CYP proteins (see below).

(c) orthologous CYP proteins

In some embodiments, the edited chromosomal sequence may comprise at least one chromosomally integrated sequence encoding an orthologous CYP protein. The orthologous CYP proteins may be from any of the animals listed above. In preferred embodiments, the orthologous CYP protein may be a human CYP protein. Additionally, the orthologous CYP protein may be a bacterial, fungal, or plant CYP protein.

In embodiments in which the orthologous CYP protein is human, the human CYP protein may be coded by PXR (NC_{—}000003.11), CAR (NC_{—}000001.10), CYP1A2 (NM_{—}000761.3), CYP2C9 (NM_{—}000771.3), CYP2C19 (NM_{—}000769.1), CYP2D6 (NC_{—}000022.10), or CYP3A4 (NM_{—}017460).

The type of animal that is genetically modified and the source of the orthologous protein can and will vary. As an example, the genetically modified animal may be rat, cat, dog, or pig, and the orthologous CYP protein may be human. Alternatively, the genetically modified animal may be rat, cat, or pig, and the orthologous CYP protein may be canine. In an exemplary embodiment, the genetically modified animal is a rat, and the orthologous CYP protein is human.

Table B lists some preferred combinations of a genetically modified rat in which at least one endogenous chromosomal sequence is inactivated and which may further comprise at least one chromosomally integrated sequence encoding an orthologous human CYP protein. For example, those rows having no entry in the “Human Ortholog” column indicate a genetically modified animal in which the sequence specified in that row under “Inactivated Rat Sequence” is inactivated (i.e., a knock-out). Subsequent rows indicate single or multiple knock-outs with knock-ins of one or more integrated orthologous sequences, as indicated in the “Human Ortholog” column. Not listed in the table are combinations in which large chromosomal regions of a rat chromosomal are deleted (see above) such that no functional homologs of the human CYP protein are made.

TABLE B

| Inactivated Rat Sequence | Human Ortholog |
|---|---|
| pxr | none |
| car | none |
| cyp1A2 | none |
| cyp2C11 | none |
| cyp2C7 | none |
| cyp2D4 | none |
| Cyp2E1 | none |
| cyp3A2 | none |
| pxr | PXR |
| car | CAR |
| cyp1A2 | CYP1A2 |
| cyp2C11 | CYP2C9 |
| cyp2C7 | CYP2C19 |
| cyp2D4 | CYP2D6 |
| cyp3A2 | CYP3A4 |
| pxr, cyp1A2 | PXR, CYP1A2 |
| pxr, cyp2C11 | PXR, CYP2C9 |
| pxr, cyp2C7 | PXR, CYP2C19 |
| pxr, cyp2D4 | PXR, CYP2D6 |
| pxr, cyp2E1 | PXR, CYP2E1 |
| pxr, cyp3A2 | PXR, CYP3A4 |
| pxr, cyp1A2, cyp2C11 | PXR, CYP1A2, CYP2C9 |
| pxr, cyp1A2, cyp2C7 | PXR, CYP1A2, CYP2C19 |
| pxr, cyp1A2, cyp2D4 | PXR, CYP1A2, CYP2D6 |
| pxr, cyp1A2, cyp2E1 | PXR, CYP1A2, CYP2E1 |
| pxr, cyp1A2, cyp3A2 | PXR, CYP1A2, CYP3A4 |
| pxr, cyp2C11, cyp2C7 | PXR, CYP2C9, CYP2C19 |
| pxr, cyp2C11, cyp2D4 | PXR, CYP2C9, CYP2D6 |
| pxr, cyp2C11, cyp2E1 | PXR, CYP2C9, CYP2E1 |
| pxr, cyp2C11, cyp3A2 | PXR, CYP2C9, CYP3A4 |
| pxr, cyp2C7, cyp2D4 | PXR, CYP2C19, CYP2D6 |
| pxr, cyp2C7, cyp2E1 | PXR, CYP2C19, CYP2E1 |
| pxr, cyp2C7, cyp3A2 | PXR, CYP2C19, CYP3A4 |
| pxr, cyp2D4, cyp3A2 | PXR, CYP2D6, CYP3A4 |
| pxr, cyp2D4, cyp2E1 | PXR, CYP2D6, CYP2E1 |
| pxr, cyp2E1, cyp3A2 | PXR, CYP2E1, CYP3A4 |
| pxr, cyp1A2, cyp2C11, cyp2C7 | PXR, CYP1A2, CYP2C9, CYP2C19 |
| pxr, cyp1A2, cyp2C11, cyp2D4 | PXR, CYP1A2, CYP2C9, CYP2D6 |
| pxr, cyp1A2, cyp2C11, cyp3A2 | PXR, CYP1A2, CYP2C9, CYP3A4 |
| pxr, cyp1A2, cyp2C7, cyp2D4 | PXR, CYP1A2, CYP2C19, CYP2D6 |
| pxr, cyp1A2, cyp2C7, cyp3A2 | PXR, CYP1A2, CYP2C19, CYP3A4 |
| pxr, cyp1A2, cyp2D4, cyp3A2 | PXR, CYP1A2, CYP2D6, CYP3A4 |
| pxr, cyp2C11, cyp2C7, cyp2D4 | PXR, CYP2C9, CYP2C19, CYP2D6 |
| pxr, cyp2C11, cyp2C7, cyp3A2 | PXR, CYP2C9, CYP2C19, CYP3A4 |
| pxr, cyp2C11, cyp2D4, cyp3A2 | PXR, CYP2C9, CYP2D6, CYP3A4 |
| pxr, cyp2C7, cyp2D4, cyp3A2 | PXR, CYP2C19, CYP2D6, CYP3A4 |
| pxr, cyp1A2, cyp2C11, cyp2C7, | PXR, CYP1A2, CYP2C9, CYP2C19, |
| cyp2D4 | CYP2D6 |
| pxr, cyp1A2, cyp2C11, cyp2C7, | PXR, CYP1A2, CYP2C9, CYP2C19, |
| cyp3A2 | CYP3A4 |
| pxr, cyp1A2, cyp2C11, cyp2D4, | PXR, CYP1A2, CYP2C9, CYP2D6, |
| cyp3A2 | CYP3A4 |
| pxr, cyp1A2, cyp2C7, cyp2D4, | PXR, CYP1A2, CYP2C19, CYP2D6, |
| cyp3A2 | CYP3A4 |
| pxr, cyp2C11, cyp2C7, cyp2D4, | PXR, CYP2C9, CYP2C19, |
| cyp3A2 | CYP2D6, CYP3A4 |
| pxr, cyp1A2, cyp2C11, cyp2C7, | PXR, CYP1A2, CYP2C9, CYP2C19, |
| cyp2D4, cyp3A2 | CYP2D6, CYP3A4 |
| car, cyp1A2 | CAR, CYP1A2 |
| car, cyp2C11 | CAR, CYP2C9 |
| car, cyp2C7 | CAR, CYP2C19 |
| car, cyp2D4 | CAR, CYP2D6 |
| car, cyp2E1 | CAR, CYP2E1 |
| car, cyp3A2 | CAR, CYP3A4 |
| car, cyp1A2, cyp2C11 | CAR, CYP1A2, CYP2C9 |
| car, cyp1A2, cyp2C7 | CAR, CYP1A2, CYP2C19 |
| car, cyp1A2, cyp2D4 | CAR, CYP1A2, CYP2D6 |
| car, cyp1A2, cyp2E1 | CAR, CYP1A2, CYP2E1 |
| car, cyp1A2, cyp3A2 | CAR, CYP1A2, CYP3A4 |
| car, cyp2C11, cyp2C7 | CAR, CYP2C9, CYP2C19 |
| car, cyp2C11, cyp2D4 | CAR, CYP2C9, CYP2D6 |
| car, cyp2C11, cyp2E1 | CAR, CYP2C9, CYP2E1 |
| car, cyp2C11, cyp3A2 | CAR, CYP2C9, CYP3A4 |
| car, cyp2C7, cyp2D4 | CAR, CYP2C19, CYP2D6 |
| car, cyp2C7, cyp2E1 | CAR, CYP2C19, CYP2E1 |
| car, cyp2C7, cyp3A2 | CAR, CYP2C19, CYP3A4 |
| car, cyp2D4, cyp3A2 | CAR, CYP2D6, CYP3A4 |
| car, cyp2D4, cyp2E1 | CAR, CYP2D6, CYP2E1 |
| car, cyp2E1, cyp3A2 | CAR, CYP2E1, CYP3A4 |
| car, cyp1A2, cyp2C11, cyp2C7 | CAR, CYP1A2, CYP2C9, CYP2C19 |
| car, cyp1A2, cyp2C11, cyp2D4 | CAR, CYP1A2, CYP2C9, CYP2D6 |
| car, cyp1A2, cyp2C11, cyp3A2 | CAR, CYP1A2, CYP2C9, CYP3A4 |
| car, cyp1A2, cyp2C7, cyp2D4 | CAR, CYP1A2, CYP2C19, CYP2D6 |
| car, cyp1A2, cyp2C7, cyp3A2 | CAR, CYP1A2, CYP2C19, CYP3A4 |
| car, cyp1A2, cyp2D4, cyp3A2 | CAR, CYP1A2, CYP2D6, CYP3A4 |
| car, cyp2C11, cyp2C7, cyp2D4 | CAR, CYP2C9, CYP2C19, CYP2D6 |
| car, cyp2C11, cyp2C7, cyp3A2 | CAR, CYP2C9, CYP2C19, CYP3A4 |
| car, cyp2C11, cyp2D4, cyp3A2 | CAR, CYP2C9, CYP2D6, CYP3A4 |
| car, cyp2C7, cyp2D4, cyp3A2 | CAR, CYP2C19, CYP2D6, CYP3A4 |
| car, cyp1A2, cyp2C11, cyp2C7, | CAR, CYP1A2, CYP2C9, |
| cyp2D4 | CYP2C19, CYP2D6 |
| car, cyp1A2, cyp2C11, cyp2C7, | CAR, CYP1A2, CYP2C9, |
| cyp3A2 | CYP2C19, CYP3A4 |
| car, cyp1A2, cyp2C11, cyp2D4, | CAR, CYP1A2, CYP2C9, CYP2D6, |
| cyp3A2 | CYP3A4 |
| car, cyp1A2, cyp2C7, cyp2D4, | CAR, CYP1A2, CYP2C19, |
| cyp3A2 | CYP2D6, CYP3A4 |
| car, cyp2C11, cyp2C7, cyp2D4, | CAR, CYP2C9, CYP2C19, |
| cyp3A2 | CYP2D6, CYP3A4 |
| car, cyp1A2, cyp2C11, cyp2C7, | CAR, CYP1A2, CYP2C9, |
| cyp2D4, cyp3A2 | CYP2C19, CYP2D6, CYP3A4 |
| pxr, car, cyp1A2 | PXR, CAR, CYP1A2 |
| pxr, car, cyp2C11 | PXR, CAR, CYP2C9 |
| pxr, car, cyp2C7 | PXR, CAR, CYP2C19 |
| pxr, car, cyp2D4 | PXR, CAR, CYP2D6 |
| pxr, car, cyp2E1 | PXR, CAR, CYP2E1 |
| pxr, car, cyp3A2 | PXR, CAR, CYP3A4 |
| pxr, car, cyp1A2, cyp2C11 | PXR, CAR, CYP1A2, CYP2C9 |
| pxr, car, cyp1A2, cyp2C7 | PXR, CAR, CYP1A2, CYP2C19 |
| pxr, car, cyp1A2, cyp2D4 | PXR, CAR, CYP1A2, CYP2D6 |
| pxr, car, cyp1A2, cyp2E1 | PXR, CAR, CYP1A2, CYP2E1 |
| pxr, car, cyp1A2, cyp3A2 | PXR, CAR, CYP1A2, CYP3A4 |
| pxr, car, cyp2C11, cyp2C7 | PXR, CAR, CYP2C9, CYP2C19 |
| pxr, car, cyp2C11, cyp2D4 | PXR, CAR, CYP2C9, CYP2D6 |
| pxr, car, cyp2C11, cyp2E1 | PXR, CAR, CYP2C9, CYP2E1 |
| pxr, car, cyp2C11, cyp3A2 | PXR, CAR, CYP2C9, CYP3A4 |
| pxr, car, cyp2C7, cyp2D4 | PXR, CAR, CYP2C19, CYP2D6 |
| pxr, car, cyp2C7, cyp2E1 | PXR, CAR, CYP2C19, CYP2E1 |
| pxr, car, cyp2C7, cyp3A2 | PXR, CAR, CYP2C19, CYP3A4 |
| pxr, car, cyp2D4, cyp3A2 | PXR, CAR, CYP2D6, CYP3A4 |
| pxr, car, cyp2D4, cyp2E1 | PXR, CAR, CYP2D6, CYP2E1 |
| pxr, car, cyp2E1, cyp3A2 | PXR, CAR, CYP2E1, CYP3A4 |
| pxr, car, cyp1A2, cyp2C11, | PXR, CAR, CYP1A2, CYP2C9, |
| cyp2C7 | CYP2C19 |
| pxr, car, cyp1A2, cyp2C11, | PXR, CAR, CYP1A2, CYP2C9, |
| cyp2D4 | CYP2D6 |
| pxr, car, cyp1A2, cyp2C11, | PXR, CAR, CYP1A2, CYP2C9, |
| cyp3A2 | CYP3A4 |
| pxr, car, cyp1A2, cyp2C7, cyp2D4 | PXR, CAR, CYP1A2, CYP2C19, |
| | CYP2D6 |
| pxr, car, cyp1A2, cyp2C7, cyp3A2 | PXR, CAR, CYP1A2, CYP2C19, |
| | CYP3A4 |
| pxr, car, cyp1A2, cyp2D4, cyp3A2 | PXR, CAR, CYP1A2, CYP2D6, |
| | CYP3A4 |
| pxr, car, cyp2C11, cyp2C7, | PXR, CAR, CYP2C9, CYP2C19, |
| cyp2D4 | CYP2D6 |
| pxr, car, cyp2C11, cyp2C7, | PXR, CAR, CYP2C9, CYP2C19, |
| cyp3A2 | CYP3A4 |
| pxr, car, cyp2C11, cyp2D4, | PXR, CAR, CYP2C9, CYP2D6, |
| cyp3A2 | CYP3A4 |
| pxr, car, cyp2C7, cyp2D4, cyp3A2 | PXR, CAR, CYP2C19, CYP2D6, |
| | CYP3A4 |
| pxr, car, cyp1A2, cyp2C11, | PXR, CAR, CYP1A2, CYP2C9, |
| cyp2C7, cyp2D4 | CYP2C19, CYP2D6 |
| pxr, car, cyp1A2, cyp2C11, | PXR, CAR, CYP1A2, CYP2C9, |
| cyp2C7, cyp3A2 | CYP2C19, CYP3A4 |
| pxr, car, cyp1A2, cyp2C11, | PXR, CAR, CYP1A2, CYP2C9, |
| cyp2D4, cyp3A2 | CYP2D6, CYP3A4 |
| pxr, car, cyp1A2, cyp2C7, | PXR, CAR, CYP1A2, CYP2C19, |
| cyp2D4, cyp3A2 | CYP2D6, CYP3A4 |
| pxr, car, cyp2C11, cyp2C7, | PXR, CAR, CYP2C9, CYP2C19, |
| cyp2D4, cyp3A2 | CYP2D6, CYP3A4 |
| pxr, car, cyp1A2, cyp2C11, | PXR, CAR, CYP1A2, CYP2C9, |
| cyp2C7, cyp2D4, cyp3A2 | CYP2C19, CYP2D6, CYP3A4 |

The genetically modified animals comprising the at least one inactivated chromosomal sequence encoding a CYP protein and, optionally, at least one chromosomally integrated sequence encoding an ortholog of the CYP protein may be generated using zinc finger nuclease technology as detailed below in section (III). Alternatively, the genetically modified animals described herein also may be generated by crossing an animal comprising at least one inactivated chromosomal sequence (i.e., a knock out animal) with an animal comprising at least one chromosomally integrated sequence (i.e., a knock in animal) as detailed below in section (III)(f).

### (II) Genetically Modified Cells

A further aspect of the present disclosure provides genetically modified cells or cell lines comprising at least one edited chromosomal sequence encoding a CYP protein. The genetically modified cell or cell line may be derived from any of the genetically modified animals disclosed herein. Alternatively, the chromosomal sequence coding a CYP protein may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

### (i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

An exemplary zinc finger DNA binding domain recognizes and binds a sequence having at least about 80% sequence identity with SEQ ID NO:2 or SEQ ID NO:3. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

In another embodiment, an exemplary zinc finger DNA binding domain may recognize and bind a sequence having at least about 80% sequence identity with a sequence listed in TABLE C. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

TABLE C

| | |
|---|---|
| DNA Binding Site | SEQ ID |
| (Contact sites in uppercase; 5′-3′) | NO: |

Cyp3A18-u

| | |
|---|---|
| tgGTAGCCaAGACAAGAActtctgctta | 4 |
| aaGTAGAAAGATATGAAataaaaggtat | 5 |
| ggTAGACGGGAGGCACAGAAggagactt | 6 |
| gtTCTCCAaGAAGTGAGAGTGgatgatg | 7 |
| gaACCAGGTAGACGGGAGGCacagaagg | 8 |
| caAGAAGTGAGAGTGGAtgatgttattt | 9 |
| ttGGAGAAcCAGGTAGACGGGaggcaca | 10 |
| aaGTGAGAGTGGATGATgttatttttct | 11 |
| gtTAAATAAGGTATGGAtagaaaaataa | 12 |
| caCATACAGAAgAGTGAAccccactggc | 13 |
| ggTTGCCTGCTGCTCAGCTGccagtggg | 14 |
| caTCATAGGCAGTGAGAAGGgggctttt | 15 |
| tgATGGGGTTGCCTGCTGCTcagctgcc | 16 |
| agGCAGTGAGAAGGGGGctttttgcagg | 17 |
| gaATAGCAGGGCAACTGacattagctca | 18 |
| tgCACCAGgGAGGGTGGAtaaccaaggg | 19 |
| gcAAGGAATAGCAGGGCaactgacatta | 20 |
| ccAGGGAGGGTGGATAAccaagggaatc | 21 |
| ccCTGGTGCAAGGAATAGCAgggcaact | 22 |
| ggGTGGATaACCAAGGGAATCaactgaa | 23 |
| taTATAGGGATgTCTGATtgataaaagc | 24 |
| aaGAAAAAGCTCCAGATgtctgaaataa | 25 |
| ctGTCCTCTAGTCTGATtttcctccaaa | 26 |
| agCAACAGATTGGAGAACTGgcaagaac | 27 |
| ttGCTGCAgTTGCCAAAAGAGttcttgc | 28 |
| ccAGACGTCTGTATGAAacacctaaaac | 29 |
| ggGCCAGGATGTATGAAcagtcatgcag | 30 |
| atCCAGTTCACCAGACCAGGtcttcaag | 31 |
| gaACTGGAtGTGGGCCAGGATgtatgaa | 32 |
| caGACCAGGTCTTCAAGagactcctctg | 33 |
| gtGTGGCTCTGCCCaTATGATctcaatg | 34 |
| ctGGTGCATCATCAGGAcaaaaagctga | 35 |

Cyp3A2-u

| | |
|---|---|
| agTCTCCATATGCGTGGGATaagataaa | 36 |
| taTAAGATAAGGGGAATGAAaaggacct | 37 |
| aaGAGTCTCCATATGCGtgggataagat | 38 |
| aaGATAAGGGGAATGAAaaggaccttta | 39 |
| taTAAGAGTCTCCATATGCGtgggataa | 40 |
| gaTAAGGGGAATGAAAAGGAcctttagt | 41 |
| atAAGGGGAATGAAAAGGACctttagtg | 42 |
| gtCAGGAGGATaAAAGCTtaagagcaac | 43 |
| ctATTGTTATGTCTCCAggttcagcaag | 44 |
| gaAGTCAGGAGGATaAAAGCTtaagagc | 45 |
| ttGTTATGTCTCCAGGTtcagcaaggag | 46 |
| aaCAATAGaAGTCAGGAGGATaaaagct | 47 |
| gtCTCCAGGTTCAGCAAGGAgaacaagt | 48 |
| caTACCCGTCTCCAATGGACttgacttg | 49 |
| caTTTGACATCCAGaGGTGCAatgatca | 50 |
| gaAGAGTCGAACATGCAacaaacattga | 51 |
| tcAGAGCACCTGGAAGGCCAtttctcat | 52 |
| ggGCAACAAGTGATTTTAGGaactgttt | 53 |
| taAAAGGAGTTGAGTATatttttcagta | 54 |
| ctCCTAAGaAATGAGCTGGAAttttact | 55 |
| ctGCTCTAGAAAAATAGgtatttgtcat | 56 |
| aaCAAGAATAGGTTATTCAGgaaaatgc | 57 |
| ctGTAAAGGAATGGGCGcatcttatagg | 58 |
| gaTGAATGGGGAAAGCCtccaatagaga | 59 |
| ctGAAGAGCACAAGGCTGAAttacaatt | 60 |
| gtGATGAATGGGGAAAGcctccaataga | 61 |
| aaGAGCACAAGGCTGAAttacaattgga | 62 |
| aaGAATGGGCAACTGATtctacaaaatt | 63 |
| caTCCTTGGAAGCCAAGaatagatgcta | 64 |
| gtGAAGAATGGGCAACTGATtctacaaa | 65 |
| ccTTGGAAGCCAAGaATAGATgctagaa | 66 |

Cyp3A23/A1-d

| | |
|---|---|
| ctCCTGTGGAAGGGaAGCTAGgagaact | 67 |
| ttAGAGCTTCCCTGATGtttgactgtgg | 68 |
| tcTAAGACtCCTGTGGAAGGGaagctag | 69 |
| ctTCCCTGATGTTTGACtgtggctctct | 70 |
| gtCGTGAATGTGGGAAGAAGatttgcag | 71 |
| gtTTCATGTCCTAAGGAcattatctagt | 72 |
| atAATGTCcTTAGGACATGAAacaagtc | 73 |
| taGTGCCATGTGCGTGACTGtgagtttg | 74 |
| cgCACATGgCACTAGATAATGtccttag | 75 |
| acTGTGAGTTTGGAAGCCTGcgggtcac | 76 |
| aaAGAGACAGGgCCTGACactcattgct | 77 |
| caCTGGCACCAAAGGGTGGCaggtgttg | 78 |
| agTGAGAAAGAGACAGGGCCtgacactc | 79 |
| caCCAAAGgGTGGCAGGTGTTgaatttt | 80 |
| ccAGTGAGAAAGAGACAGGGcctgacac | 81 |
| acCAAAGGGTGGCAGGTGTTgaattttg | 82 |
| tgCCAGTGAGAAAGAGACAGggcctgac | 83 |
| aaAGGGTGGCAGGTGTTGAAttttggtg | 84 |
| ggTGCCAGTGAGAAAGAGACagggcctg | 85 |
| agGGTGGCaGGTGTTGAAttttggtgga | 86 |
| ttGGTGCCAGTGAGAAAGAGacagggcc | 87 |
| ggGTGGCAGGTGTTGAAttttggtggat | 88 |
| ccAGCACGCATGATgAGTGCTtctttgt | 89 |
| caGTTGCCTCAGGGGTTGAGtgggcttt | 90 |
| tgTCCAGCACGCATGATgagtgcttctt | 91 |
| tgCCTCAGGGGtTGAGTGggcttttgca | 92 |
| ccAGAAGTGCAAAAGCCcactcaacccc | 93 |
| aaCTGGTGCTTGATTCCtagacattaca | 94 |
| tcTAGGAAtCAAGCACCAGTTctccaga | 95 |
| ttACAGTGGAGATATAGgtccttgagca | 96 |
| caGTGCCATATAAGTAAGACcaaagttt | 97 |
| gtTCACTGaCGCCACCCAGAAtgttaac | 98 |

Cyp2A1

| | |
|---|---|
| aaCAGAAGATGGCAGTGGCCagtagcag | 99 |
| gtGTTCTGGGTGTTgagaggcacaagga | 100 |
| acTAAACAGAAGATGGCagtggccagta | 101 |
| tcTGGGTGtTGAGAGgCACAAGgaccca | 102 |
| ctTCAGACcTTTGGGAACCTGggtcctt | 103 |
| tcCTCCCGGACCCTGGGgcttgcccttc | 104 |
| gaGGATACTGATGTGGGgtctgaggcta | 105 |
| gcTACTTGGAGGAGCACGTGagcaaaga | 106 |
| aaGAGGATACTGATGTGGGGtctgaggc | 107 |
| taCTTGGAGGAGCAcgtgagcaaagagg | 108 |
| agCAAGAGGATACTGATgtggggtctga | 109 |
| ctTGGAGGAGCACGTGAGCAaagaggct | 110 |
| taGCAAGAGGATACTGAtgtggggtctg | 111 |
| tgGAGGAGCACGTGAGCAAAgaggctaa | 112 |
| acTTGCTGaTTAGATGGTtagcctcttt | 113 |
| caGAAGCTGATGGCAGAGGTtggccact | 114 |
| ttGACTGGTTCGAAGTGGCCaacctctg | 115 |
| caGGTGGTGGAATCGGTGGCtaatgtca | 116 |
| gtTGACTGgTTCGAAGTGGCCaacctct | 117 |
| agGTGGTGGAATCGGTGGCTaatgtcat | 118 |
| acCACCTGGTTGACtggttcgaagtggc | 119 |
| aaTCGGTGGCTAATGTCatcggagccat | 120 |
| ttCACGAGGTTGAGcatctcctcgctct | 121 |
| gcAGCAAGGACTTTGTGgagaatgtcac | 122 |
| ggGTTGGGCAGGTAGCGCAGgaccggaa | 123 |
| ccCTCAAGAGGTTTAAGaacttcaatga | 124 |
| gcTGGGTTGGGCAGgtagcgcaggaccg | 125 |
| ctCAAGAGGTTTAAGAActtcaatgata | 126 |
| agGGCTGGGTTGGGCAGGTAgcgcagga | 127 |
| aaGAGGTTTAAGAActtcaatgataact | 128 |
| gtCTTGATAGTGTTCCTGGActgttttc | 129 |
| caACAAGGTGAGACTGAGAGgcagactg | 130 |

Cyp2C-u

| | |
|---|---|
| tcTGTGTGAGGGTCTCAGAAgctgtgga | 131 |
| taCATGGAGGCAAAACAAGGttgaacta | 132 |
| agAATGCGgGATAACAGAATGgtcataa | 133 |
| gcTAAAAGgGCTTTGTATgcaaaaggta | 134 |
| agAGAATGCGGGATAACagaatggtcat | 135 |
| ctAAAAGGGCTTTGTATgcaaaaggtac | 136 |
| gcAGAGAAtGCGGGATAAcagaatggtc | 137 |
| aaAGGGCTTTGTATgCAAAAGgtacctt | 138 |
| aaGTCATGTAATTAGCTcatataataaa | 139 |
| ttGGTGAGTAATCATGGctaatattact | 140 |
| ttGGTGAGTAATCATGGctaatattact | 141 |
| caAGAGCCtGGCTTGCCAtaaaaatgac | 142 |
| tgGATGTTGCTATGTAACAGttgggtag | 143 |
| caGACAAGAGCCTGGCTtgccataaaaa | 144 |
| atGTTGCTaTGTAACAGTTGGgtagagc | 145 |
| taGCAACATCCaGACAAGagcctggctt | 146 |
| taACAGTTGGGTAGagccaagaaggaat | 147 |
| taACAGTTGGGtAGAGCCaagaaggaat | 148 |
| acCCAAAGTTAGCTtcttagcattcctt | 149 |
| caCCCCTGgTTAGTTACACAGatttgtg | 150 |
| ggGTGGGAcCCAAAGTTAGCTtcttagc | 151 |
| ggTTAGTTACACAGaTTTGTGgtagtag | 152 |
| taACCAGGGGTGGGacccaaagttagct | 153 |
| taACCAGGGGTGGGACCcaaagttagct | 154 |
| ttACACAGaTTTGTGGTAGTAgaaagag | 155 |
| caAATCTGTGTAACtaaccaggggtggg | 156 |
| ggTAGTAGAAAGAGGAAGAAcagttttc | 157 |
| ccACAAATCTGTGTAACtaaccaggggt | 158 |
| gtAGAAAGaGGAAGAACAGTTttcactt | 159 |

Cyp2C-d

| | |
|---|---|
| ggGGAACATAGgCCAGAAggaaaccggg | 160 |
| aaTGTGATGGTATTGCAtgtgggacagg | 161 |
| gaGGGGAACATAGGCCAGAAggaaaccg | 162 |
| atGTGATGGTATTGCATGTGggacaggc | 163 |
| caTTGAGGGGAACATAGgccagaaggaa | 164 |
| atGGTATTGCATGTGGGacaggcataag | 165 |
| ctCTAGAAATGAGTGCAgagagttctgg | 166 |
| ggGAAACAGCCATTGGActcccctccag | 167 |
| gtCCAATGGCTGTTTCCcctctctagaa | 168 |
| ccCTCCAGGTTCCTGACtccccctgctg | 169 |
| caGGAACCTGGAGGGGAgtccaatggct | 170 |
| tcCCCCTGCTGGCAGTAtaccagcggtg | 171 |
| gaGTCAGGAACCTGGAGGGGagtccaat | 172 |
| ccCTGCTGGCAGTATACCAGcggtgcct | 173 |
| caGAGAGGAAGCAGAGCAAAgtctgaaa | 174 |
| ggCATGCCAGCGGCCCTTGGttacaccc | 175 |
| ggGGGGGTGTAACCAAGggccgctggca | 176 |
| caCCCCTGGTAGAGAGGCAGgatgtagc | 177 |
| ggGTGGGGGGGGGGTGTAACcaagggcc | 178 |
| tgGTAGAGAGGCAGGATGTAgccctata | 179 |
| agGGGTGGGGGGGGGGTGTAaccaaggg | 180 |
| gtAGAGAGGCAGGAtGTAGCCctatatg | 181 |
| acCAGGGGTGGGGGGGGGGTgtaaccaa | 182 |
| agAGGCAGGATGTAGCCctatatgcaat | 183 |
| tcTACCAGGGGTGGGGGGGGggtgtaac | 184 |
| ggCAGGATGTAGCCcTATATGcaatctc | 185 |
| ccCAAGATGTGCAGAAACTGaccagaga | 186 |
| ttTGTGCAtTATGCAAGAGGAtgggaaa | 187 |
| ccTTAGAGACAATAGACtaactctgcac | 188 |
| aaTAAAAAGCTCATAGGaacaacacaca | 189 |
| gtGGCAGAGCTCTGGAAcatggcttcat | 190 |
| tgGAGGTTtTTTGGAGTCagcctgggat | 191 |

Cyp2D-u

| | |
|---|---|
| acAGGGATGATTCGGTTCAGagtaaaac | 192 |
| gcTCTCCAGGCTGTAAGgggcctgagca | 193 |
| ctCACAGGGATGATTCGGTTcagagtaa | 194 |
| tcTCCAGGCTGTAAGGGgcctgagcagc | 195 |
| ggAGAGCTCACAGGGATGATtcggttca | 196 |
| gcTGTAAGGGGCCTGAGcagccttcccg | 197 |
| caCATGGTCAAGGAAGAttaagtagggg | 198 |
| ccAATGGAAGGGCTGCTctactgacctc | 199 |
| ggGCACATGGTCAAGGAagattaagtag | 200 |
| atGGAAGGGCTGCTcTACTGAcctccga | 201 |
| ccATTGGGgCACATGGTCAAGgaagatt | 202 |
| ggGCTGCTcTACTGACCTCCGaaatggc | 203 |
| tcCAGATGTTGGCATCTATGaataaaca | 204 |
| ggAGGGCCAGAAAGGACtgctgtgaagg | 205 |
| ttCCAGATgTTGGCATCTATGaataaac | 206 |
| agGGCCAGAAAGGACTGCTGtgaagggt | 207 |
| gtGGGTCTGAAATGGGGgcgtctgggag | 208 |
| acACAACCAAGGCTAACtcctcagccag | 209 |
| gtAGTGGGTCTGAAATGGGGgcgtctgg | 210 |
| caACCAAGGCTAACTCCtcagccagcat | 211 |
| aaGTTGTGATGATGCTGGCTgaggagtt | 212 |
| taTATGACGTCGCAGAGATGtagagaag | 213 |
| aaGAAGTTGTGATGATGCTGgctgagga | 214 |
| atGACGTCGCAGAGATGtagagaagtcg | 215 |
| taTAAGAAGTTGTGATGatgctggctga | 216 |
| acGTCGCAGAGATGtAGAGAAgtcgggg | 217 |
| taGGGAAGCATGTGGTAcccaacaggta | 218 |
| acGGTTTTGTGGGGGTCcagaagcaggt | 219 |
| aaACCGTTTAGGGAAGCATGtggtaccc | 220 |
| gtGGGGGTCCAGAAGCAGGTtgcctcct | 221 |
| atGGTGACAAAGAAGCTTAGgaggcaac | 222 |
| atTCCATGACCCAGCAGGGAtactggtg | 223 |

Cyp2D-d

| | |
|---|---|
| ctTTAGGGGGGATGGCAtctgcatattt | 224 |
| caCATGATCCTGACAGGCCAcaggggtt | 225 |
| gtGACTTTAGGGGGGATggcatctgcat | 226 |
| gaTCCTGACAGGCCACAGGGgttcccat | 227 |
| ctGTGGCCTGTCAGGATCATgtgacttt | 228 |
| gtTCCCATGGATGGGAGgacatggactg | 229 |
| acAGGTGGAAGGTCGTTcaagtctcaag | 230 |
| ttGAAGCTTCACAGGCTGGAgttacctt | 231 |
| aaGTACAGGTGGAAGGTcgttcaagtct | 232 |
| aaGCTTCACAGGCTGGAgttaccttcct | 233 |
| ccACTGAAGTGGCTTGTGTGggtagctg | 234 |
| ccTTGCTGcATACTGATAGTGgccatgg | 235 |
| caAGGGCCACTGAAGTGGCTtgtgtggg | 236 |
| gcATACTGATAGTGGCCATGgtgtcagt | 237 |
| atGCAGCAAGGGCCACTGAAgtggcttg | 238 |
| tgATAGTGGCCATGGTGtcagtcagacc | 239 |
| acTATCAGTATGCAGCAAGGgccactga | 240 |
| gcCATGGTGTCAGTCAGaccccatgtct | 241 |
| gcCTTATGGGCAGAGATccacggggcct | 242 |
| atCAGCAGCTTGCAcACTGAGgaagggt | 243 |
| tgCAAGCTGCTGATTTGCCTtatgggca | 244 |
| acTGAGGAAGGGTTCATGTTatgtgctt | 245 |
| tcAGTGTGCAAGCTGCTGATttgcctta | 246 |
| gaAGGGTTCATGTTaTGTGCTtgtcttg | 247 |
| gtGGCATTGAAaTCTCCAcagaaagcag | 248 |
| ccCATGGACAAGCACCACCGgagagaag | 249 |
| tgTCCATGGGAGTGGCAttgaaatctcc | 250 |
| caCCACCGgAGAGAAGAAAGGgcaagaa | 251 |
| gtGGTGCTTGTCCATGGGAGtggcattg | 252 |
| ggAGAGAAGAAAGGgCAAGAAcctctga | 253 |
| ccGGTGGTGCTTGTCCAtgggagtggca | 254 |
| gaGAAGAAAGGGCAAGAacctctgatgt | 255 |

### (ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from Ito K in one cleavage monomer to produce an engineered cleavage monomer designated “E490K:1538K” and by mutating positions 486 from Q to E and 499 from I to L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences encoding CYP proteins may further comprise introducing at least one donor polynucleotide comprising a sequence encoding an orthologous CYP protein into the embryo or cell. A donor polynucleotide comprises at least three components: a sequence to be inserted in the target site, which may be the sequence coding the CYP protein ortholog, an upstream sequence, and a downstream sequence. The sequence to be inserted is flanked by the upstream and downstream sequence, wherein the upstream and downstream sequences share sequence similarity with either side of the site of integration in the chromosome.

Typically, the donor polynucleotide will be DNA. The donor polynucleotide may be single or double stranded, a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding an orthologous CYP protein may be a BAC.

The sequence of the donor polynucleotide that encodes the orthologous CYP protein may include coding (i.e., exon) sequence, as well as intron sequences and upstream regulatory sequences (such as, e.g., a promoter). Depending upon the identity and the source of the orthologous CYP protein, the size of the sequence encoding the CYP protein can and will vary. For example, the sequence encoding the CYP protein may range in size from about 1 kb to about 5,000 kb. In some embodiments, the sequence encoding the orthologous CYP protein may be fused to a sequence encoding a reporter protein. Reporter proteins include without limit cloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a donor polynucleotide comprising a reporter sequence, the reporter gene sequence may be fused directly to the CYP sequence to create a gene fusion. A reporter sequence may be integrated in a targeted manner in the CYP sequence, for example the reporter sequences may be integrated specifically at the 5′ or 3′ end of the CYP sequence.

In another embodiment, donor polynucleotides may be used to introduce recombination sites into the two or more locations in a cyp chromosomal region or cyp gene cluster such that introduction of the corresponding recombinase may result in deletion of the intervening chromosomal region. For example loxP sites may be introduced into cyp chromosomal regions such that expression of Cre recombinase may lead to deletion a large chromosomal regions,

The donor polynucleotide also comprises upstream and downstream sequence flanking the sequence encoding the orthologous CYP protein. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 by to about 500 kb. In one embodiment, an upstream or downstream sequence may range from about 100 by to about 1 kb, from about 1 kb to about 10 kb, from about 10 kb to about 100kb, or from about 100 kb to about 500 kb.

In some embodiments, the donor polynucleotide may further comprise a marker. Such a marker may make it easy to screen for targeted integrations. Non-limiting examples of suitable markers include restriction sites, reporter proteins (see above), or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a sequence encoding the CYP protein, a double stranded break introduced into the chromosomal sequence by the zinc finger nuclease is repaired, via homologous recombination with the donor polynucleotide, such that the sequence encoding the orthologous CYP protein is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the sequence encoding the CYP protein as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences encoding CYP protein may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 by to about 10,000 by in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 by in length. In other embodiments, the sequence in the exchange polynucleotide may be about 5500, 6000, 6500, 6000, 6500, 7000, 7500, 8000, 8500, 9000, 9500, or 10,000 by in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence encoding the CYP protein in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or point mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

### (f) Crossbreeding

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated pxr chromosomal sequence may be crossed with animal B comprising a chromosomally integrated sequence encoding a human PXR protein to give rise to a “humanized” PXR offspring comprising both the inactivated pxr chromosomal sequence and the chromosomally integrated human PXR sequence. Similarly, an animal comprising an inactivated cyp1A2 chromosomal sequence may be crossed with an animal comprising a chromosomally integrated sequence encoding the human CYP1A2 protein to generate “humanized” CYP1A2 offspring. Moreover, a humanized PXR animal may be crossed with a humanized CYP1A2 animal to create a humanized PXR/CYP1A2 animal. Those of skill in the art will appreciate that many combinations are possible. Exemplary combinations are presented above in TABLE B.

In other embodiments, additional cyp chromosomal sequences may be edited by introducing at least one targeted zinc finger nucleases into embryos from animals already carrying one or more edited chromosomal sequences encoding CYP proteins.

In still embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with another targeted integration, and genetic backgrounds with non-targeted integrations. Suitable integrations may include without limit nucleic acids encoding drug transporter proteins, Mdr protein, and the like.

### (IV) Applications

A further aspect of the present disclosure encompasses a method for assessing the effect(s) of an agent. Suitable agents include without limit pharmaceutically active ingredients, biologics, therapeutic agents, diagnostic agents, drugs, food additives, pesticides, herbicides, toxins, industrial chemicals, household chemicals, and other environmental chemicals. For example, the effect(s) of an agent may be measured in a “humanized” genetically modified animal, such that the information gained therefrom may be used to predict the effect of the agent in a human. In general, the method comprises contacting a genetically modified animal comprising at least one inactivated chromosomal sequence encoding a CYP protein and at least one chromosomally integrated sequence encoding an orthologous CYP protein with the agent, and comparing results of a selected parameter to results obtained from contacting a wild-type animal with the same agent.

Selected pharmacodynamic or pharmacokinetic parameters include but are not limited to (a) rate of elimination of the agent or its metabolite(s); (b) circulatory levels of the agent or its metabolite(s); (c)bioavailability of the agent or its metabolite(s); (d) rate of metabolism of the agent or its metabolite(s); (e) rate of clearance of the agent or its metabolite(s); (f) toxicity of the agent or its metabolite(s); (g) efficacy of the agent or its metabolite(s); (h) disposition of the agent or its metabolite(s); (i) extrahepatic contribution to metabolic rate and clearance of the agent or its metabolite(s); (j) potential drug-drug interactions; and (k) potential drug-other substance interactions.

Also provided are methods to assess the effect(s) of an agent in an isolated cell comprising at least one edited chromosomal sequence encoding a CYP protein, as well as methods of using lysates of such cells (or cells derived from a genetically modified animal disclosed herein) to assess the effect(s) of an agent. For example, the role of a particular CYP protein in the metabolism of a particular agent may be determined using such methods. Similarly, substrate specificity and pharmacokinetic parameter may be readily determined using such methods. Those of skill in the art are familiar with suitable tests and/or procedures.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Identification of ZFNs that Edit the Pxr Locus in Rat Cells

The chromosomal sequence encoding PXR chosen for zinc finger nuclease (ZFN) mediated genome editing. ZFNs were designed, assembled, and validated using strategies and procedures previously described (see Geurts et al. Science (2009) 325:433). ZFN design made use of an archive of pre-validated 1-finger and 2-finger modules. The rat pxr gene region (NM-052980) was scanned for putative zinc finger binding sites to which existing modules could be fused to generate a pair of 4-, 5-, or 6-finger proteins that would bind a 12-18 by sequence on one strand and a 12-18 by sequence on the other strand, with about 5-6 by between the two binding sites.

Capped, polyadenylated mRNA encoding each pair of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′-caTACACGGCAGATTTGaagacctccat-3′ (SEQ ID NO:2) and 5′-ggGACAAGGCCaATGGCTatcacttcaa-3′ (SEQ ID NO:3) edited the pxr locus.

### Example 2

### Editing the Pxr Locus

Capped, polyadenylated mRNA encoding the active pair of ZFNs was microinjected into fertilized rat embryos using standard procedures (e.g., see Geurts et al. (2009) supra). The injected embryos were transferred to pseudopregnant female rats to be carried to parturition. The resulting embryos/fetus, or the toe/tail clip of live born animals were harvested for DNA extraction and analysis. DNA was isolated using standard procedures. The targeted region of the pxr locus was PCR amplified using appropriate primers. The amplified DNA was subcloned into a suitable vector and sequenced using standard methods. FIG. 1 presents the DNA sequence of an edited pxr locus. There was a one base pair deletion within the target sequence in exon 2 of the Pxr coding region. This one base pair deletion disrupts the reading frame of the Pxr coding region.

The table below presents the amino acid sequences of helices of the active ZFNs.

| | | SEQ ID |
|---|---|---|
| Name | Sequence of Zinc Finger Helices | NO: |
| PXR | DNAALTE TSSNLSR QSGDLTR RSDTLSQ DNANRTK | 256 |
| PXR | QSSDLSR RSDALTQ DRSDLSR RSDNLSV DRSNLTR | 257 |

### Example 3

### Identification of ZFNs that Edit cyp Chromosomal Regions

ZFNs that target cyp gene clusters in rat may be designed such that large chromosomal regions can be deleted. For example, the rat cyp2D cluster may be deleted by targeting ZFN pairs to upstream or downstream regions of the cyp2D cluster. Thus, introduction of upstream and downstream ZFN pairs may lead to deletion of the region between the two targeted regions. For this, ZFNs may be designed and tested as detailed above in Example 1. Active ZFN pairs may be introduced into rat embryos as described above in Example 2, wherein a large chromosomal region may be deleted. Other rat cyp gene clusters may be targeted using a similar strategy. Such genetically modified rats may be crossed with rats comprising at least one chromosomally integrated sequences encoding a human CYP protein to generate “humanized” rats.

What is claimed is:

1. A genetically modified animal comprising at least one edited chromosomal sequence encoding a CYP protein.

2. The genetically modified animal of claim 1, wherein the at least one edited chromosomal sequence is inactivated, modified, or comprises an integrated sequence.

3. The genetically modified animal of claim 1, wherein the edited chromosomal sequence is inactivated such that no functional CYP protein is produced.

4. The genetically modified animal of claim 3, wherein the inactivated chromosomal sequence comprises a deletion of a cluster of sequences encoding CYP proteins.

5. The genetically modified animal of claim 3, wherein the inactivated chromosomal sequence comprises no exogenously introduced sequence.

6. The genetically modified animal of claim 3, further comprising at least one chromosomally integrated sequence encoding an ortholog of the CYP protein.

7. The genetically modified animal of claim 1, wherein the at least one CYP protein is a functional homolog of human PXR, CAR, CYP1A2, CYP2C9, CYP2C19, CYP2D6, CYP2E1, CYP3A4, and combinations thereof.

8. The genetically modified animal of claim 1, wherein the animal is heterozygous or homozygous for the at least one edited chromosomal sequence.

9. The genetically modified animal of claim 1, wherein the animal is an embryo, a juvenile, or an adult.

10. The genetically modified animal of claim 1, wherein the animal is chosen from bovine, canine, equine, feline, ovine, porcine, non-human primate, and rodent.

11. The genetically modified animal of claim 6, wherein the animal is a rat and the orthologous CYP protein is human.

12. A non-human embryo, the embryo comprising at least one RNA molecule encoding a zinc finger nuclease that recognizes a site in a chromosomal sequence encoding a CYP protein, and, optionally, at least one donor polynucleotide comprising a sequence encoding an ortholog of the CYP protein.

13. The non-human embryo of claim 12, wherein the CYP protein is a functional homolog of human PXR, CAR, CYP1A2, CYP2C9, CYP2C19, CYP2D6, CYP2E1, or CYP3A4.

14. The non-human embryo of claim 12, wherein the embryo is chosen from bovine, canine, equine, feline, ovine, porcine, non-human primate, and rodent.

15. The non-human embryo of claim 12, wherein the embryo is rat and the orthologous CYP protein is human.

16. A genetically modified cell, the cell comprising at least one edited chromosomal sequence encoding a CYP protein.

17. The genetically modified cell of claim 16, wherein the at least one edited chromosomal sequence is inactivated, modified, or comprises an integrated sequence.

18. The genetically modified cell of claim 16, wherein the edited chromosomal sequence is inactivated such that no functional CYP protein is produced.

19. The genetically modified cell of claim 18, wherein the inactivated chromosomal sequence comprises a deletion of a cluster of sequences encoding CYP proteins.

20. The genetically modified cell of claim 18, wherein the inactivated chromosomal sequence comprises no exogenously introduced sequence.

21. The genetically modified cell of claim 18, further comprising at least one chromosomally integrated sequence encoding an ortholog of the CYP protein.

22. The genetically modified cell of claim 16, wherein the at least one CYP protein is a functional homolog of human PXR, CAR, CYP1A2, CYP2C9, CYP2C19, CYP2D6, CYP2E1, CYP3A4, and combinations thereof.

23. The genetically modified cell of claim 16, wherein the cell is heterozygous or homozygous for the at least one edited chromosomal sequence.

24. The genetically modified cell of claim 16, wherein the cell is of bovine, canine, equine, feline, human, ovine, porcine, non-human primate, or rodent origin.

25. The genetically modified cell of claim 21, wherein the cell is of rat origin and the orthologous CYP protein is human.

26. A zinc finger nuclease, the zinc finger nuclease comprising:
a) a zinc finger DNA binding domain that binds a sequence having at least about 80% sequence identity with SEQ ID NO:2 or SEQ ID NO:3; and
b) a cleavage domain.

27. The zinc finger nuclease of claim 26, wherein the sequence identity is at least about 85%, 90%, 95%, or 100%.

28. The zinc finger nuclease of claim 26, wherein the DNA binding domain comprises at least three zinc finger recognition regions.

29. The zinc finger nuclease of claim 26, wherein the cleavage domain is a wild-type or an engineered FokI cleavage domain.

30. A nucleic acid sequence bound by a zinc finger nuclease, the nucleic acid sequence having at least 80% sequence identity with SEQ ID NO:2 or SEQ ID NO:3.

31. The nucleic acid sequence of claim 30, wherein the sequence identity is at least about 85%, 90%, 95%, or 100%.

32. A method for assessing the effect of an agent in an animal, the method comprising contacting a genetically modified animal comprising at least one edited chromosomal sequence encoding a CYP protein with the agent, and comparing results of a selected pharmacodynamic or pharmacokinetic parameter to results obtained from contacting a wild-type animal with the same agent, wherein the selected pharmacodynamic or pharmacokinetic parameter is chosen from:
a) rate of elimination of the agent or its metabolite(s);
b) circulatory levels of the agent or its metabolite(s);
c) bioavailability of the agent or its metabolite(s);
d) rate of metabolism of the agent or its metabolite(s);
e) rate of clearance of the agent or its metabolite(s);
f) toxicity of the agent or its metabolite(s);
g) efficacy of the agent or its metabolite(s);
h) potential drug-drug interactions; and
i) potential drug-other substance interactions.

33. The method of claim 32, wherein the agent is a pharmaceutically active ingredient, a biologic, a therapeutic agent, a diagnostic agent, a drug, a toxin, or a chemical.

34. The method of claim 32, wherein the at least one CYP protein is a functional homolog of human PXR, CAR, CYP1A2, CYP2C9, CYP2C19, CYP2D6, CYP2E1, CYP3A4, and combinations thereof.

35. The method of claim 32, wherein the at least one edited chromosomal sequence is inactivated such that no functional CYP protein is produced, and wherein the animal further comprises at least one chromosomally integrated sequence encoding an ortholog of the CYP protein.

36. The method of claim 35, wherein the animal is a rat and the orthologous CYP protein is human.
